(19) United States
(12) Patent Application Publication
(10) Pub. No.: US 2017/0239150 A1
(43) Pub. Date: Aug. 24, 2017

(54) FACIAL TISSUE SYSTEMS

(71) Applicant: Jennipher Pennella, Toronto (CA)

(72) Inventor: Jennipher Pennella, Toronto (CA)

(21) Appl. No.: 15/051,878

(22) Filed: Feb. 24, 2016

(51) Int. Cl. A61K 8/02; B32B 27/10; B32B 27/30; B65D 83/08

(52) U.S. Cl. CPC A61K 8/0208; B65D 83/0835; B32B 27/10; B32B 27/306; B32B 2307/7163; B32B 2555/00

(57) ABSTRACT

A facial tissue system for the purpose of eliminating the spread of bacteria when sneezing or blowing nose into a tissue. The facial tissue system has a two-sided design; a soft textile side, and an eco-safe plastic durable side; however, the tissue remains flushable and septic safe. The eco-safe plastic side provides strength to the structure of the tissue in order to prevent tears and mucus pass-through during usage while also being ecological.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of tissues (and wipes) and more specifically relates to a facial tissue system.

2. Description of Related Art

Many people suffer from sinus obstruction for various reasons. Nasal mucus contains bacterium that can spread when sneezing or blowing one's nose. Once contagion occurs, it can easily be spread by touching other commonly used objects such as doorknobs, office equipment, public phones, public transportation, within hospitals or medical offices, shopping carts and the like. When expelling nasal mucus using a conventional tissue, mucus passes through the tissue which contaminates the hands. Also, during a severe sneeze, mucus may puncture conventional thinly designed tissues rendering the tissue useless. Since conventional two-ply tissue is so ‘thinly’ designed, users are accustomed to using a multitude of tissues in order to deal with the common occurrence of rupturing a single tissue. It is therefore desirable for a tissue to be designed which not only avoids contamination of hands, but also is more durable and environmentally friendly.

Several attempts have been made to solve the above-mentioned problems such as those found in U.S. and International Pub. And Pat. Nos. AU 734408 to Dean Van Phan et al, CA 2162360 to Dean Van Phan et al, CA 2205649 to Paul Thomas Weisman et al, EP 0795057 to Dean Van Phan et al, JP 2003034372 to Ueda Atsuko et al, JP 2005162259 to Nishitani Chiemi et al, U.S. Pat. No. 5,527,560 to Saeed Fereshtehkhou et al, U.S. Pat. No. 5,981,044 to Dean Van Phan et al, U.S. Pat. No. 6,146,365 to Jimmy Phong Xuan Nguyen, and WO 2013137791. This art is representative of conventional tissues. However, none of the above inventions and patents, taken either singly or in combination, is seen to describe the invention as claimed.

Preferably, a facial tissue system should provide a durable tissue which greatly lowers contamination and, yet would operate reliably and be manufactured at a modest expense. Thus, a need exists for a reliable facial tissue system to avoid the above-mentioned problems.

### BRIEF SUMMARY OF THE INVENTION

In view of the foregoing disadvantages inherent in the known tissue art, the present invention provides a novel facial tissue system. The general purpose of the present invention, which will be described subsequently in greater detail is to provide a facial tissue system that is durable, yet environmentally-friendly for use.

A facial tissue system is disclosed herein, in a preferred embodiment, comprising: a facial tissue assembly comprising a textile front surface, a defined thickness, and a thinly coated plastic rear surface preferably comprising a biodegradable and/or compostable cover as a backing. The facial tissue system comprises the facial tissue assembly, wherein the facial tissue assembly is defined by the textile front surface, the thinly coated plastic rear surface, and the thickness of the textile front surface provides an absorbent, and disposable volume for wiping. In preferred embodiments the usage of polyvinyl alcohol (PVA) plastic for the biodegradable cover allows the facial tissue to be disposed of in a traditional commode.

The facial tissue assembly comprises in integral functional combination, a textile front surface, a thinly coated plastic rear surface, and a thickness that provides the textile front surface. The textile front surface and the thinly coated plastic rear surface are separated by the thickness, yet all are integrally formed during manufacture. The facial tissue assembly is structured and arranged for providing a barrier to block bacterium from passing through the thickness of the textile front surface and contaminating a hand of a user when expelling nasal mucus in an effort to promote public health.

The textile front surface may comprise pure chemical pulp. The pure chemical pulp may comprise recycled fiber. The thinly coated plastic rear surface comprises ecological plastic; the ecological plastic comprises polyvinyl alcohol (PVA) which is biodegradable, as mentioned previously. The textile front surface and the thinly coated plastic rear surface comprise a width which may be at least eight inches. The textile front surface and the thinly coated plastic rear surface comprise a length which is at least eight inches. The facial tissue device may comprise exactly two-ply (other versions with more or less plys may be used). For ease of shipping and use the facial tissue assembly may be housed in a dispenser such as a box or the like.

A kit is also described herein including: the facial tissue assembly, the dispenser, and a set of user directions.

A method of use for a facial tissue system is also disclosed herein comprising the steps of: opening the dispenser, placing the facial tissue assembly in a palm of a hand, using the facial tissue assembly to expel nasal mucus, and disposing of the contaminated facial tissue assembly after use in an environmentally-friendly manner. The method may further comprise the steps of: flushing the contaminated facial tissue down a commode, and sealing the dispenser to protect the remaining facial tissue assemblies.

The present invention holds significant improvements and serves as a facial tissue system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, facial tissue system, constructed and operative according to the teachings of the present invention.

FIG. 1 shows a perspective view illustrating a facial tissue system during an ‘in-use’ condition according to an embodiment of the present invention.

FIG. 2A is a perspective view illustrating the facial tissue system according to an embodiment of the present invention of FIG. 1.

FIG. 2B is another perspective view illustrating the facial tissue system according to an embodiment of the present invention of FIG. 1.

FIG. 3 is another perspective view illustrating components of a facial tissue assembly according to an embodiment of the present invention of FIGS. 1-2B.

FIG. 4 is a perspective view illustrating the facial tissue system according to an embodiment of the present invention of FIGS. 1-3.

FIG. 5 is a flowchart illustrating a method of use for the facial tissue system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a tissue and more particularly to an facial tissue system as used to lower the risk of contamination due to airborne bacterium when sneezing or expelling mucus into such a tissue.

Generally speaking, the facial tissue system preferably comprises a two-ply system composed of a textile side and an eco-friendly plastic side. The textile side is soft to the touch to avoid chaffing of the nose and face while the eco-friendly plastic side gives the tissue durability to avoid tearing the tissue while sneezing or blowing nose. The advantages of having the eco-friendly plastic side are the ability to use less tissues since they are more durable, the protection from contamination, and a product which is more economical since there is no longer a need to grab handfuls of tissues. By using environmentally friendly plastic, such as polyvinyl alcohol (PVA) plastic, the user is also protecting the environment.

Preferred embodiments include pure chemical pulp, and recycled fiber for the textile side, and an environmentally friendly plastic side. Certain embodiments may include the environmentally friendly plastic side comprised of polyvinyl alcohol (PVA), or other similar biodegradable material. The components of the facial tissue system allow for disposal in an environmentally and septic safe manner down a conventional commode. The textile side in certain embodiments also comprise but are not limited to lotion, fragrance, and mentholated topical petrolatum-based gel. The preferred embodiment design includes a dispenser for the purpose of storage.

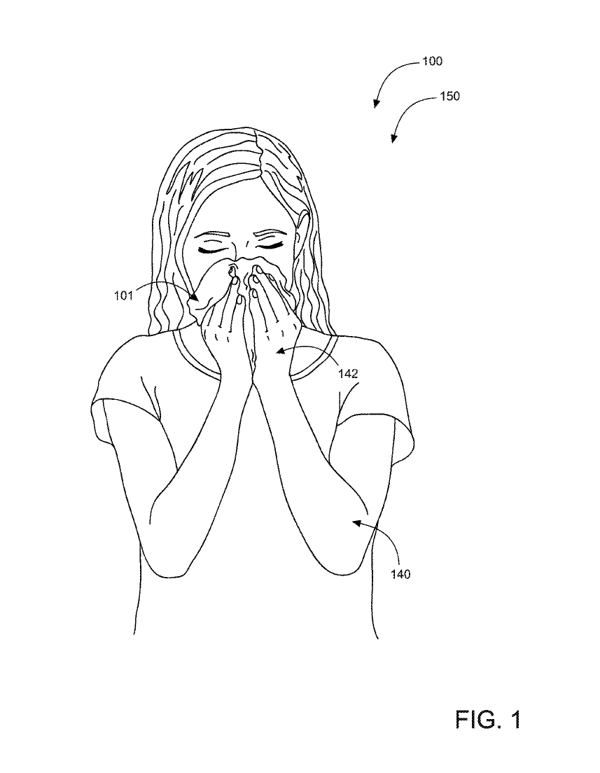

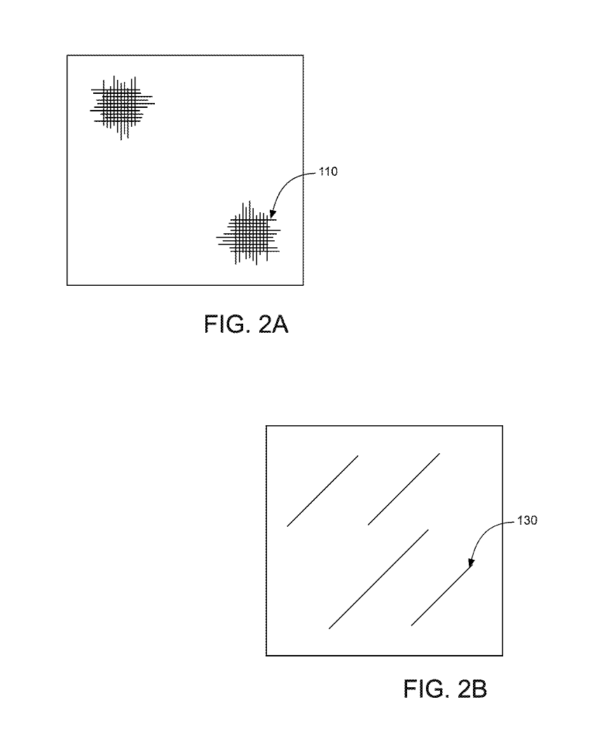

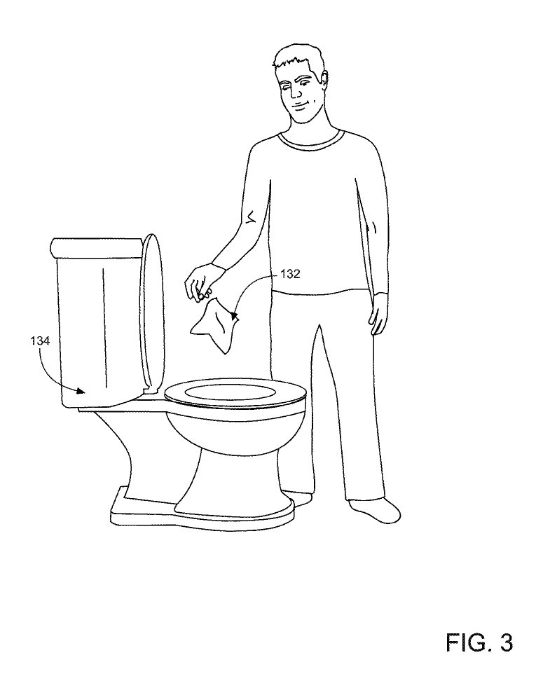

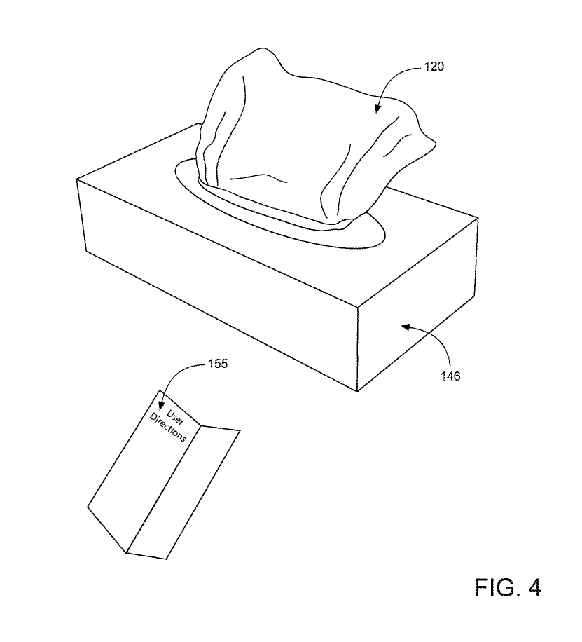

Referring to the drawings by numerals of reference there is shown in FIGS. 1-3, facial tissue system 100 comprising: facial tissue assembly 101 comprising textile front surface 110, thickness 120; and thinly coated plastic rear surface 130, comprising biodegradable cover 132 as backing. Textile front surface 110 preferably comprises pure chemical pulp; pure chemical pulp 112 comprising recycled fiber 114.

Thinly coated plastic rear surface 130 comprises ecological plastic. Textile front surface 110 and thinly coated plastic rear surface 130 comprises width 118; width 118 comprises at least eight inches, but may be more or less as per application. Textile front surface 110 and thinly coated plastic rear surface 130 comprises length 119; length 119 comprises at least eight inches, but may be more or less as per application.

Facial tissue assembly 101 may comprise exactly two-ply(s). In certain embodiments facial tissue assembly 101 may comprises more than two-ply(s). Facial tissue assembly 101 is able to be housed in dispenser 146 before use. In certain embodiments facial tissue assembly 101 may comprises lotion, a fragrance, and mentholated topical petrolatum-based gel. Facial tissue system 100, having a biodegradable composition, allows facial tissue assembly 101 to be discarded down commode 134.

Referring now again to facial tissue system 100; facial tissue system 100 comprises facial tissue assembly 101; wherein facial tissue assembly 101 is defined by textile front surface 110, thinly coated plastic rear surface 130, thickness 120 of textile front surface 110, in combination providing an absorbent, and disposable volume for wiping.

Facial tissue assembly 101 comprises in integral functional combination textile front surface 110 and thinly coated plastic rear surface 130; thickness 120 providing integrally textile front surface 110. Textile front surface 110 and thinly coated plastic rear surface 130 are separated by thickness 120, yet all integrally formed. As such facial tissue assembly 101 is structured and arranged for providing a barrier to block bacterium from passing through thickness 120 of textile front surface 110 and contaminating hand 142 of a user 140 when expelling nasal mucus to promote public health.

Facial tissue system 100 may be sold as kit 440 comprising the following parts: at least one facial tissue assembly 101; at least one dispenser 146; and at least one set of user directions 155. The kit has instructions such that functional relationships are detailed in relation to the structure of the invention (such that the invention can be used, maintained, or the like in a preferred manner). Facial tissue system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components, customized parts, different color combinations, parts may be sold separately, etc., may be sufficient.

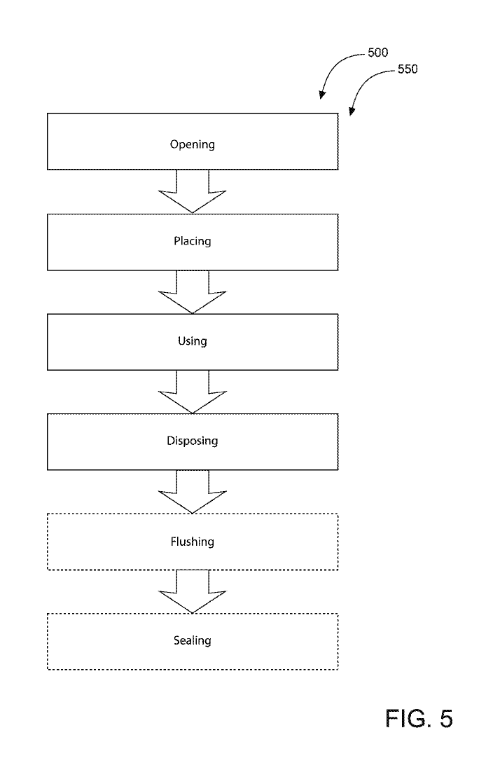

Referring now to FIG. 5 showing flowchart 550 illustrating method of use 500 for facial tissue system 100 according to an embodiment of the present invention of FIGS. 1-3.

As shown, method of use 500 may comprise the steps of: step one 501, opening dispenser 146; step two 502, placing facial tissue assembly 101 in a palm of hand 142; step three 503, using facial tissue assembly 101 to expel nasal mucus; step four 404, disposing of contaminated facial tissue assembly 101 after use in an environmentally friendly manner. The method 500 may further comprise the steps of; step five 505, flushing contaminated facial tissue assembly 101 down commode 134; and step six 506, sealing dispenser 146 to protect remaining facial tissue assemblies 101.

It should be noted that step 505 and 506 are optional steps and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. §112, ¶ 6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A facial tissue system comprising:
a facial tissue assembly comprising;
a textile front surface having;
a thickness; and
a thinly coated plastic rear surface comprising a biodegradable cover as a backing;
wherein said facial tissue system comprises said facial tissue assembly;
wherein said facial tissue assembly is defined by said textile front surface, said thinly coated plastic rear surface, said thickness of said textile front surface providing an absorbent, and disposable volume for wiping;
wherein said facial tissue assembly comprises in integral functional combination said textile front surface and said thinly coated plastic rear surface, said thickness providing integrally said textile front surface, and said thinly coated plastic rear surface and said thinly coated plastic rear surface are separated by said thickness, yet all integrally formed; and
wherein said facial tissue assembly is structured and arranged for providing a barrier to block bacterium from passing through said thickness of said textile front surface and contaminating a hand of a user when expelling nasal mucus to promote public health.

2. The facial tissue system of claim 1 wherein said textile front surface comprises pure chemical pulp.

3. The facial tissue system of claim 2 wherein said pure chemical pulp comprises recycled fiber.

4. The facial tissue system of claim 1 wherein said thinly coated plastic rear surface comprises ecological plastic which is biodegradable and compostable.

5. The facial tissue system of claim 4 wherein said ecological plastic comprises polyvinyl alcohol (PVA) which is biodegradable.

6. The facial tissue system of claim 1 wherein said textile front surface and said thinly coated plastic rear surface comprises a width.

7. The facial tissue system of claim 6 wherein said width comprises at least eight inches.

8. The facial tissue system of claim 1 wherein said textile front surface and said thinly coated plastic rear surface comprises a length.

9. The facial tissue system of claim 8 wherein said length comprises at least eight inches.

10. The facial tissue system of claim 1 wherein said facial tissue assembly comprises exactly two-ply.

11. The facial tissue system of claim 1 wherein said facial tissue assembly comprises more than two-ply.

12. The facial tissue system of claim 10 wherein said facial tissue assembly is housed in a dispenser before use.

13. The facial tissue system of claim 11 wherein said facial tissue assembly comprises fragrance.

14. The facial tissue system of claim 13 wherein said thickness of said facial tissue assembly comprises lotion.

15. The facial tissue system of claim 1 wherein said thickness of said facial tissue assembly comprises mentholated topical petrolatum-based gel.

16. The facial tissue system of claim 1 wherein said facial tissue assembly, having a biodegradable composition, allows said facial tissue assembly to be discarded down a commode.

17. A facial tissue system comprising:
a facial tissue assembly comprising;
a textile front surface having;
a thickness; and
a thinly coated plastic rear surface comprising a biodegradable and compostable cover as a backing;
wherein said facial tissue system comprises said facial tissue assembly;
wherein said facial tissue assembly is defined by said textile front surface, said thinly coated plastic rear surface, said thickness of said textile front surface providing an absorbent, and disposable volume for wiping;
wherein said facial tissue assembly comprises in integral functional combination said textile front surface and said thinly coated plastic rear surface, said thickness providing integrally said textile front surface, and said thinly coated plastic rear surface and said thinly coated plastic rear surface are separated by said thickness, yet all integrally formed;
wherein said textile front surface comprises pure chemical pulp;
wherein said pure chemical pulp comprises recycled fiber;
wherein said thinly coated plastic rear surface comprises ecological plastic which is biodegradable and compostable;
wherein said ecological plastic comprises high density polyethylene which is biodegradable;
wherein said textile front surface and said thinly coated plastic rear surface comprises a width;
wherein said width comprises at least eight inches;
wherein said textile front surface and said thinly coated plastic rear surface comprises a length;
wherein said length comprises at least eight inches;
wherein said facial tissue assembly comprises exactly two-ply;
wherein said facial tissue assembly is housed in a dispenser before use;
wherein said facial tissue assembly is structured and arranged for providing a barrier to block bacterium from passing through said thickness of said textile front surface and contaminating a hand of a user when expelling nasal mucus to promote public health;

18. The facial tissue system of claim 17 further comprising a kit including:
said facial tissue assembly;
said dispenser; and
a set of user directions.

19. A method of using a facial tissue system comprising the steps of:
opening a dispenser;
placing a facial tissue assembly in a palm of a hand;
using said facial tissue assembly to expel nasal mucus thereon; and
disposing of contaminated said facial tissue assembly after use in an environmentally-friendly manner.

20. The method of claim 19 further comprises the steps of:
flushing contaminated said facial tissue assembly down a commode; and
sealing said dispenser to protect remaining said facial tissue assemblies.
